(19) United States
(12) Patent Application Publication
(10) Pub. No.: US 2011/0112365 A1
(43) Pub. Date: May 12, 2011

(54) Endoscope shaft

(75) Inventors: Nison Galperin, Fairfield, CT (US); Lawrence James St. George, Sudbury, MA (US); Gregory S. Konstorum, Stamford, CT (US); Kurt G. Shelton, Woburn, MA (US)

(73) Assignee: Gyrus ACMI, Inc.

(21) Appl. No.: 12/928,142

(22) Filed: Dec. 2, 2010

Related U.S. Application Data
(63) Continuation-in-part of application No. 12/455,642, filed on Jun. 3, 2009.
Application No. 12/455,642 is a continuation-in-part of application No. 12/456,986, filed on Jun. 24, 2009.

(51) Int. Cl. A61B 1/00

(52) U.S. Cl. 600/118

(57) ABSTRACT

An endoscope including a control section; and a shaft extending from the control section. The shaft includes a frame having a one-piece tube, The tube includes at least one slot into the tube to form spaced sections on opposite sides of the slot. A first one of the sections includes at least one projection which extends into at least one pocket of a second one of the sections such that the projection and pocket form an over-travel limiter to limit relative motion of the first and second sections relative to each other in at least one direction.

### CROSS REFERENCE TO RELATED APPLICATION

This application is a continuation-in-part patent application of U.S. application Ser. No. 12/455,642 filed Jun. 3, 2009, and U.S. application Ser. No. 12/456,986 filed Jun. 24, 2009, which are hereby incorporated by reference in its entireties.

### BACKGROUND OF THE INVENTION

1. Field of the Invention

The invention relates to an endoscope and, more particularly, to a shaft of an endoscope.

2. Brief Description of Prior Developments

U.S. Pat. No. 6,749,560 B1, which is hereby incorporated by reference in its entirety, discloses a endoscope shaft having a tube comprises of a superelastic material and straight slots. U.S. Pat. No. 6,485,411 B1, which is hereby incorporated by reference in its entirety, discloses an endoscope shaft having a tube comprised of a superelastic material and a single spiral slot.

### SUMMARY

The following summary is merely intended to be exemplary. The summary is not intended to limit the scope of the claimed invention.

In accordance with one embodiment of the invention, an endoscope is provided including a control section; and a shaft extending from the control section. The shaft includes a frame having a one-piece tube, The tube includes at least one slot into the tube to form spaced sections on opposite sides of the slot. A first one of the sections includes at least one projection which extends into at least one pocket of a second one of the sections such that the projection and pocket form an over-travel limiter to limit relative motion of the first and second sections relative to each other in at least one direction.

In accordance with another embodiment of the invention, an endoscope shaft frame member is provided comprising a one-piece tube, wherein the tube comprises at least one slot into the tube, wherein one of the slots has a non-straight shape to form at least one projection formed by the slot which extends into at least one pocket formed by the slot such that upon axial twist deformation of the tube the at least one projection is adapted to contact the at least one pocket to form an over-travel limiter to limit the axial twist deformation of the tube.

In accordance with another embodiment of the invention, a method comprises providing a one-piece tube; and making at least one slot into the tube to form at least one section of the tube with an increased flexibility, wherein the at least one slot comprises a slot having a non-straight shape to form a projection formed by the slot which extends into a pocket formed by the slot such that the projection and pocket form an over-travel limiter to limit axial twist deformation of the tube.

### BRIEF DESCRIPTION OF THE DRAWINGS

The foregoing aspects and other features of the invention are explained in the following description, taken in connection with the accompanying drawings, wherein:

FIG. 1 is a side elevational view of an endoscope;

FIG. 2 is a cross-sectional view of the shaft of the endoscope shown in FIG. 1;

FIG. 3 is a side elevational view of the tube used for the frame of the shaft shown in FIG. 2;

FIG. 4 is an enlarged perspective view of a portion of the tube shown in FIG. 3;

FIG. 5 is a side view of a portion of the tube shown in FIGS. 3-4 showing the tube bent;

FIG. 6 is a side view of a distal end of an alternate embodiment of an endoscope without its outer cover;

FIG. 7 is an enlarged perspective view of a portion of the distal end shown in FIG. 6;

FIG. 8 is a cross sectional illustration of an alternate embodiment of the twist limiter projection shown in FIG. 4;

FIG. 9 is a cross sectional illustration of another alternate embodiment of the twist limiter projection shown in FIG. 4;

FIG. 10 is a plan top illustration of another alternate embodiment of the twist limiter projection and pocket shown in FIG. 4;

FIG. 11 is a plan top illustration of another alternate embodiment of the twist limiter projection and pocket shown in FIG. 4;

FIG. 12 is a plan top illustration of another alternate embodiment of the twist limiter projection and pocket shown in FIG. 4;

FIG. 13 is a perspective view of a portion of an alternate embodiment of the tube used as part of the shaft frame of the tool shown in FIG. 1;

FIG. 14 is an enlarged view of a portion of the tube shown in FIG. 13;

FIG. 15 is an enlarged view of one of the pairs or the projections and pockets formed by the slot shown in FIG. 14;

FIG. 16 is a perspective view of an alternate embodiment of the tube used as part of the shaft frame of the tool shown in FIG. 1;

FIG. 17 is an enlarged view of a portion of the tube shown in FIG. 16 showing one of the pairs or the projections and pockets formed by the slot shown in FIG. 16;

FIG. 18 is a perspective view of a portion of an alternate embodiment of the tube used as part of the shaft frame of the tool shown in FIG. 1;

FIG. 19 is a perspective view of a portion of another alternate embodiment of the tube used as part of the shaft frame of the tool shown in FIG. 1; and

FIG. 20 is a side view of the tube shown in FIG. 19.

### DETAILED DESCRIPTION OF EMBODIMENTS

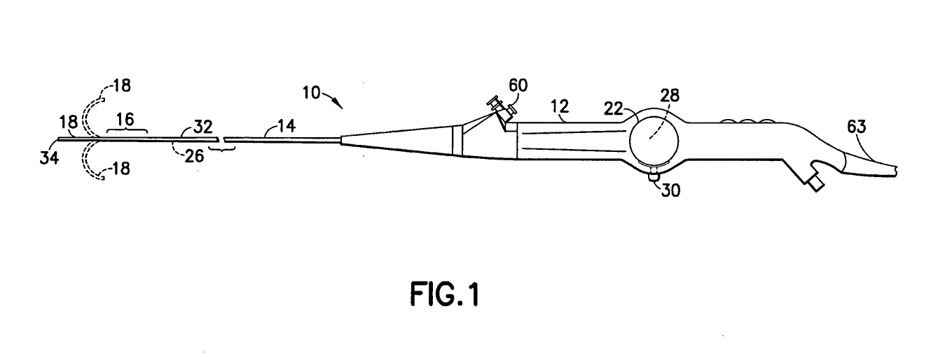

Referring to FIG. 1, there is shown a side view of an endoscope 10. Although the invention will be described with reference to the example embodiments shown in the drawings, it should be understood that the invention can be embodied in many alternate forms of embodiments. In addition, any suitable size, shape or type of elements or materials could be used.

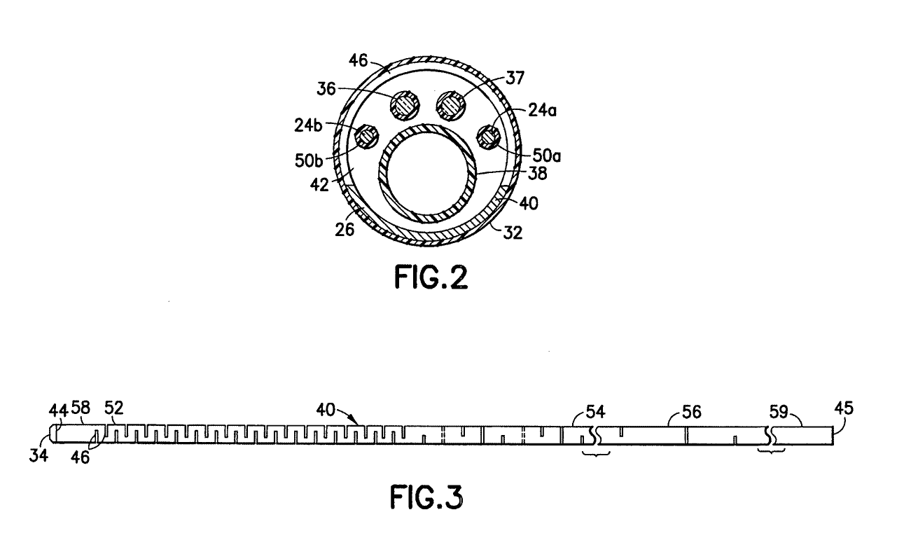

The endoscope 10 is a ureteroscope. However, in alternate embodiments the endoscope could be any suitable type of endoscope. The endoscope 10 generally comprises a handle or control 12 and a flexible or semi-flexible shaft 14 connected to the handle 12. The shaft 14 includes a passive deflection section 16 and an active deflection section 18 at the distal end of the shaft 14. A control system 22 to control the active deflection section 18 extends from the handle 12 to the active deflection section 18. Referring also to FIG. 2, the control system 22 generally comprises a pair of control wires 24a, 24b or at least one control wire, two wire sheaths 50a, 50b, and an actuator 28. The wires 24a, 24b are connected to the actuator 28 at one end and are connected to the active deflection section 18 at a second end.

In the preferred embodiment, the handle 12 has a user operated slide or lever 30. The lever 30 is connected to the actuator 28. The actuator 28 is adapted to pull and release the two wires 24a, 24b of the control system 22. When the lever 30 is moved by the user, the actuator 28 is moved. The actuator 28 may be a drum or pulley rotatably connected to the handle 12 to pull one wire 24a, 24b while optionally releasing the other. In an alternate embodiment, the actuator may be any suitable type of device, such as a rocker arm adapted to pull and release the wires of the control system 22. In another alternate embodiment, where the control system may have two or more pairs of control wires, the handle will have additional actuators and corresponding controls to drive the additional pairs of control wires. In still other alternate embodiments, the handle may have knobs with rack and pinion mechanisms or other suitable user operated controls for the control system.

The shaft 14 is cantilevered from the handle 12. The flexible shaft 14 includes the control wires 24a, 24b of the control system 22, a fiber optical image bundle 37 or sensor cable, at least one fiber optical illumination bundle 36, and a working channel 38. A port 60 for inserting instruments (not shown) into the channel 38 is located on the handle 12. In addition, the handle 12 has an electrical cable 63 for connection to another device, such as a video monitor. In an alternate embodiment, instead of the cable 63, the endoscope could have an eyepiece. In alternate embodiments, the flexible shaft may house different systems within.

The shaft 14 generally comprises a frame 26, a cover 32 and an objective head 34. Referring also to FIG. 3, the frame 26 generally comprises a one-piece tube 40. However, in alternate embodiments the frame could be comprised of more than one tube, such as multiple tubes connected in series, and could comprise additional members. The tube 40 is preferably comprised of a shape memory alloy material, such as Tinel or Nitinol. The shape memory alloy material is used for its superelastic properties exhibited by the material's ability to deflect and resiliently return to its natural or predetermined position even when material strains approach 4%, or an order of magnitude greater than the typical yield strain of 0.4% giving rise to plastic deformation in common metals. Thus, the term “superelastic alloy” is used to denote this type of material. However, tube 40 can use any durable material. The wire sheaths 50a, 50b may also be comprised of this type of material such as disclosed in U.S. Pat. No. 5,938,588 which is hereby incorporated by reference in its entirety. In an alternate embodiment the tube might not be comprised of a superelastic alloy.

The tube 40 has a center channel 42 with open front and rear ends 44, 45, and slots 46 along at least part of its length. In this embodiment the slots 46 extend more than half way through the tube. However, in alternate embodiments one or more of the slots might not extend more than half way through the tube. In this embodiment the slots have different patterns along different sections or lengths of the tube. More specifically, in this embodiment the slots 46 are configured into three sections 52, 54, 56. Each section has a different pattern of the slots 46. The pattern(s) of the slots 46 can be configured based upon, for example, the following variables:
distance or spacing between adjacent slots;
direction(s) of the slots into the tube 40;
depth of the slots into the tube;
width of the slots;
shape of the slots; and
intermixing of different directions of the slots along a length of the tube.

In alternate embodiments the tube 40 could have more or less than three sections of different slot patterns, such as only one or two for example. In addition, rather than abrupt transitions between sections of different slot patterns, the tube could be provided with gradual or intermixed slot transition zones between sections. In this embodiment the tube 40 also has two sections 58, 59 which do not have slots therein.

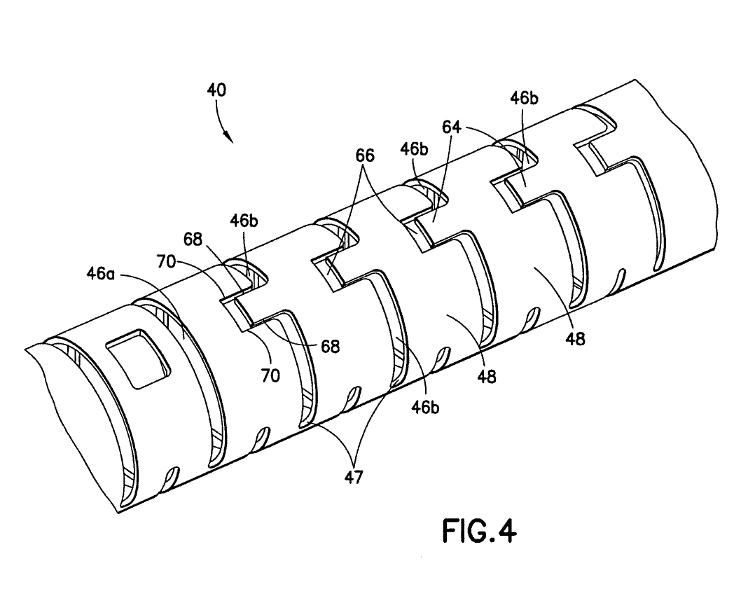

Referring also to FIG. 4, an enlarged view of a front end of the tube 40 is shown. The slots 46 include first slots 46a and second slots 46b. The first slots 46a are substantially straight, and extend into the tube generally perpendicular to the center longitudinal axis of the tube 40. The second slots 46b have a non-straight shape. In this example embodiment the second slots 46b have a general three-dimensional curved general zigzag shape. This shape forms projections 64 and pockets 66. The slots form spaced sections 48 on opposite sides of each slot 46b, wherein a first one of the sections comprises one of the projections 64 which extends into the pocket 66 of an opposite second one of the sections 48. Each second slot 46b has opposite ends 47 on opposite sides of the tube, which are aligned and generally perpendicular to a center axis of the tube. The first slots 46a, because they are straight, do not have the pockets and projections.

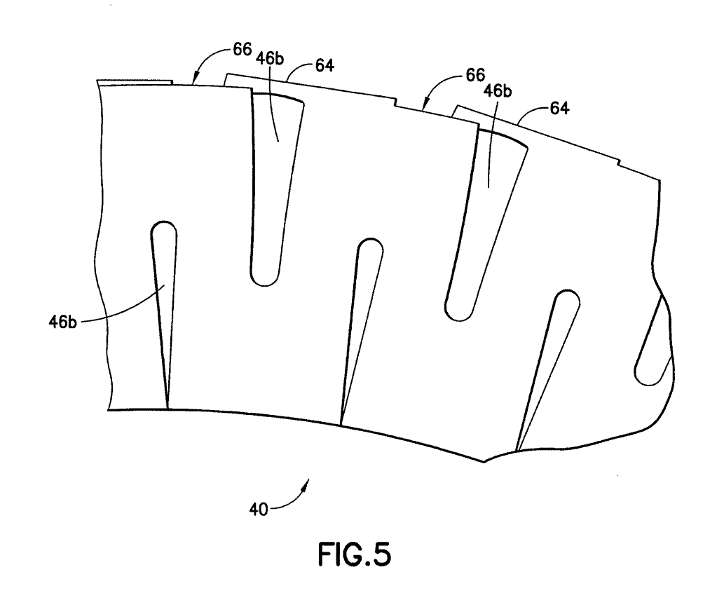

Referring also to FIG. 5, the slots 46 allow the tube 40 to bend. The projections 64 can longitudinally slide forward and backward in the pockets 66 during this bending. Lateral sides 68 of the projections 64 are normally slightly spaced from lateral sides 70 of the pockets 66. However, if the tube 40 encounters an axial torque or twisting force, the sides 68, 70 can contact each other and limit twisting of the adjacent sections 48 relative to each other. Thus, the projections and pockets form an over-travel limiter to limit relative motion of the first and second sections relative to each other in at least one direction. In this particular example the limiter limits axial twisting or deformation of the tube 40.

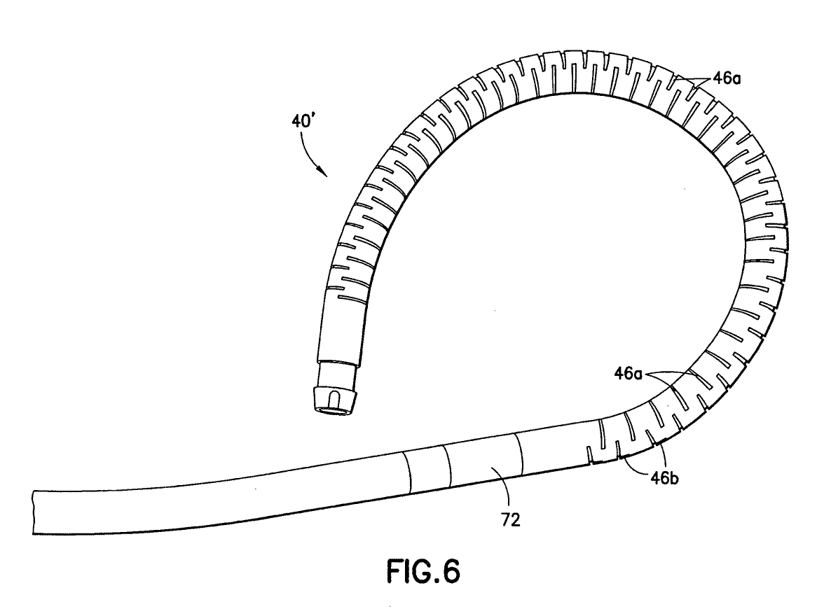

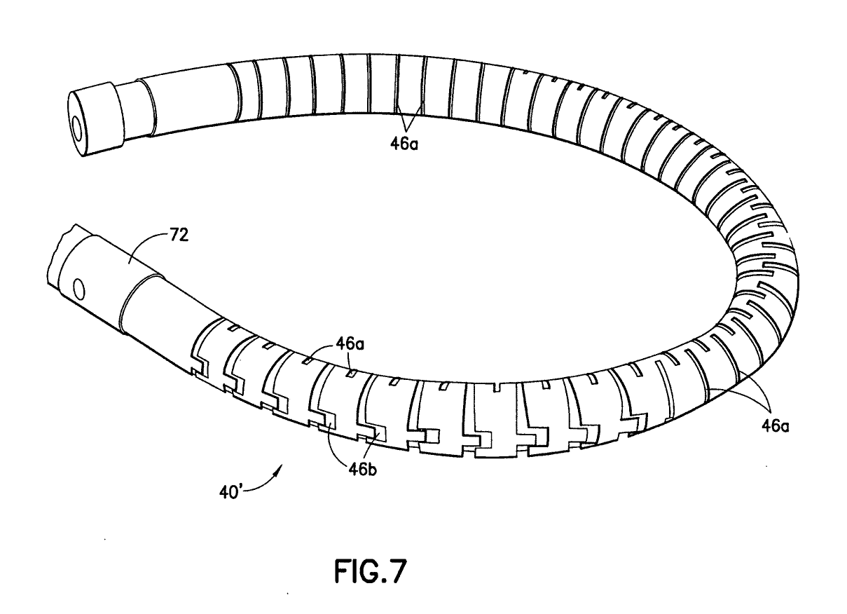

FIGS. 6 and 7 shown an alternate embodiment of the invention wherein the tube 40′ is provided only at the distal end of the shaft (the outer cover of the shaft is not shown merely for the sake of understanding). In this example embodiment the second slots 46b are merely provided at a rear section of the tube 40′ proximate a junction 72 with the rest of the shaft. In addition, the second slots 46b are merely provided at one side of the tube 40′. The first slots 46a are on the other side of the tube, interleaved with the second slots 46b, and located in front of the second slots 46b on the same side. Any suitable arrangement of the first and second slots 46a, 46b relative to each other could be provided. Additional differently shaped slots could also be provided, or the tube might only have the second slots 46b.

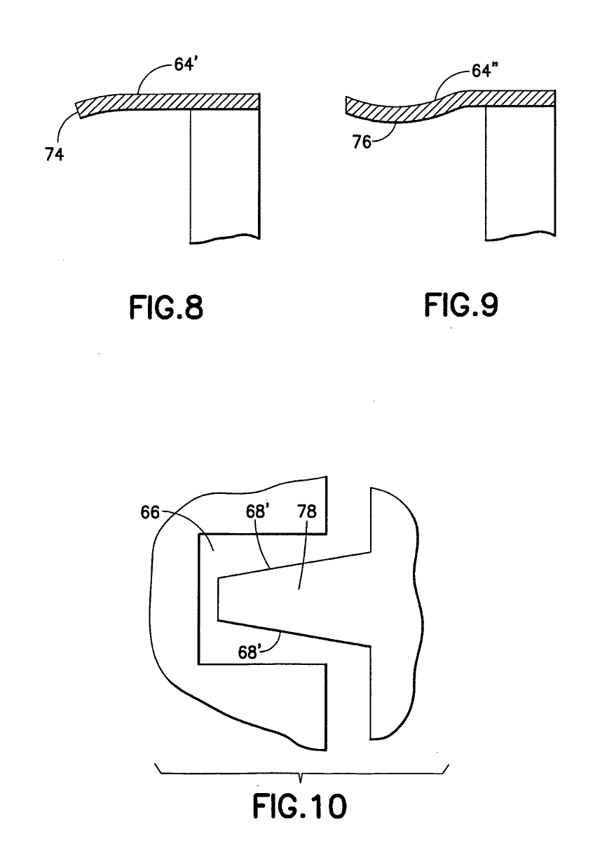

FIG. 4 shows the projection 64 as a general cantilevered rectangular shape. However, one or more of the projections 64 could have a different shape. FIG. 8 illustrates a projection 64′ with an inwardly shaped tip 74. FIG. 9 illustrates a projection 64″ with an inwardly shaped middle 76. FIG. 10 illustrates a projection 78 in a pocket 66 wherein the projection has sloped lateral sides 68′. Depending upon the longitudinal position of the projection 78 in the pocket (such as based upon the amount of bend of the tube), the amount of axial twist allowed can be varied with this embodiment.

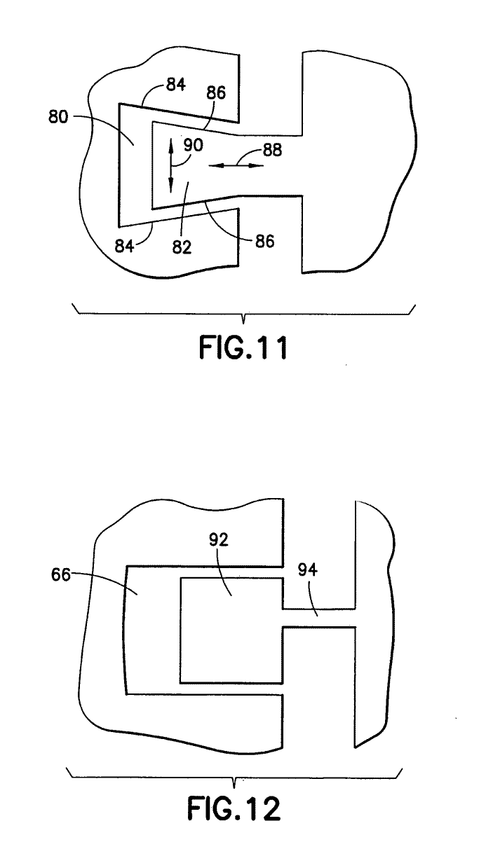

FIG. 11 illustrates another embodiment wherein the shapes of the pocket 80 and projection 82 can be used to limit longitudinal motion 88 (when the lateral sides 84, 86 wedge against each other); in addition to limiting the amount of axial twist (relative motion in direction 90). This can limit the amount of bending of the tube.

FIG. 12 illustrates another embodiment wherein the projection 92 has a resiliently deflectable spring section 94 to provide a spring action to the over-travel limiter.

With the invention, a method can be provided comprising providing a tube of superelastic alloy; and making a plurality of slots into the tube to form at least one section of the tube with an increased flexibility, wherein the slots each have a non-straight shape to form a projection which extends into a pocket and can longitudinally move relative to the pocket but has limited lateral movement in the pocket, such that the projection and pocket form an over-travel limiter to limit axial twist deformation of the tube. The method of making the slots can include, for example, laser forming of the slots in the tube.

Conventional endoscopes having a tube frame member comprising a superelastic alloy with slots perpendicular to deflections plane are known as noted above. Geometry of these slots corresponds to the requirements needed in the deflection elasticity. Slotted tubes, in some cases made from laser-cut tubing, have been used in the active deflection portion of flexible ureteroscopes with good success for a number of years. Generally, the slotted tubes have been designed to deflect in one direction, or opposing directions, and the length of the slotted tubes at maximum has been on the order of about two inches.

Newer designs of endoscopes have been using longer slotted tubes with similar defection capability in two opposing directions, but these longer version slotted tubes have shown some propensity to break at the proximal end of the tube. The present understanding is that the longer slotted tube is more likely to experience a higher torque force (than the shorter slotted tubes in earlier designs) in the proximal end as the endoscope tip at the distal end is being manipulated to the sides during a medical procedure (twisted). The earlier designs seem to have had more flexibility in the proximal end of the endoscope's deflection section, whereas deflection sections utilizing a longer slotted tube (about 3 inches long) do not have such proximal section flexibility. This stronger torque force can strongly twist and deform the proximal section of the long slotted tube and, this deformation can lead to material fatigue despite the use of superelastic material as the frame of the slotted tube. Existing slotted tube frame members work well with deflection loads, but cannot withstand angular loads (torque) because higher “deflection flexibility”, lower “torque resistance stability”.

With the longer slotted tubes noted above, the proximal end of the slotted tube (prior to the bend) seems to absorb the twist, with some prominent bend lines showing from the bottom of the open slots into the adjacent slots in that area, and the tube construction did not seem to allow the twist to propagate to the tip.

One of the purposes of the invention is to reduce the deformation of the material of the proximal section of the slotted tube due to a strong twistings and, thus, eliminate a large source of material fatigue. A basic difference of the proposed design is that the rings (sections 48) between the slots have protrusions or tabs at the center of the slot, directed along the axis of the slotted tube, and associated notches on the following coil (section 48) of the tube. The protrusion or tab 64 can function as a key. The locations of the pockets 66 are perpendicular to the plane of deflection, in order to improve the durability of the slotted tube. The solution is intended to resolve the physical contradiction of higher deflections flexibility and lower torque resistance stability. Implementation of the proposed slotted tube key design will not only increase the tube torque resistance, it will also make the slotted tube more stable in the deviation from bending plane (skew).

If twisted, the rings/coils in a conventional slotted tube frame member could and would shift transversely relative to each other; causing the web of material between adjacent slots to deform and perhaps creases form at sites where the tube material would experience stress. With the invention on the other hand, when the section with interlocking tabs (keys) is twisted, the tabs transfer the twisting force onto the next ring (section 48) with very little relative transverse displacement. This virtually eliminates the excessive material deformation and associated excessive stress. The tab 64 extends into the adjacent slit 66 enough so that when the slotted tube deflects there is still engagement of tab to slot. Tab (key) geometry may be varied to allow for variations in overall tube design, but a fundamental purpose is preserved; to translate the twisting force to the next ring (section 48) with a minimal amount of relative transverse displacement between existing sections 48 and, thus, a minimal amount of material deflection and associated stress.

The one-piece tube 40 allows the shaft to be assembled much easier than a tube comprised of multiple links or rings connected by pins or rivets. Quality control is also much more uniform for a one-piece tube than for multiple links or rings connected by pins or rivets. However, unlike other one-piece tubes used as a shaft frame in an endoscope, the torsional over-travel limiter provided by the projections/pockets of the example embodiments can allow a one piece tube to be used without the need for additional torsional stability by adding additional components. The cover no longer has to provide torque stability as needed in a conventional endoscope shaft with a one-piece tube frame. Thus, the shaft 14 can be thinner than a conventional endoscope shaft with a one-piece tube frame.

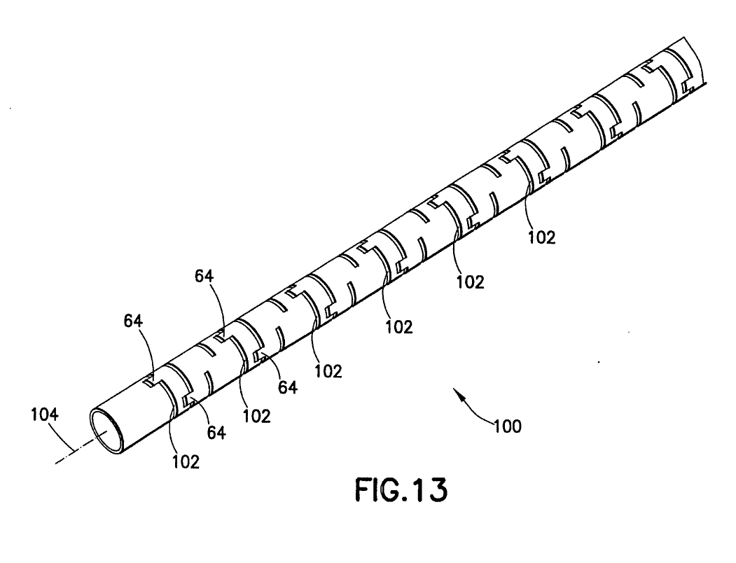

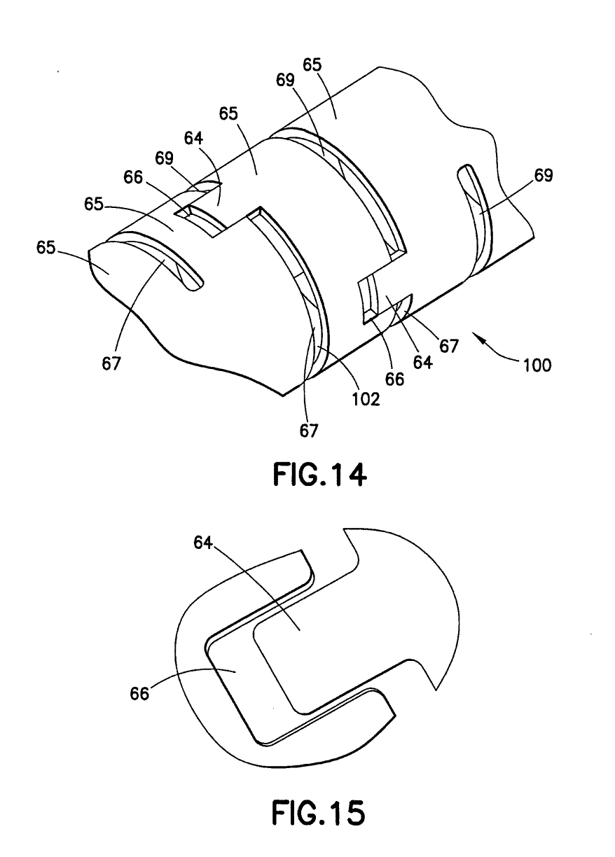

Referring now to FIGS. 13-15, another alternate embodiment of the tube used for at least part of the frame of the endoscope 10 is shown. The tube 100 is a one piece member comprised of a suitable material such as plastic, metal or metal alloy for example. The tube 100 has multiple slots 102 therein. FIG. 14 shows an enlarged view of a portion of the tube 100, and FIG. 15 shows an enlarged view of one of the pairs of projection/pocket 64/66. Each slot 102 forms one projection 64 and one pocket 66 as well as portions 67, on opposite sides of the pair of projection/pocket 64/66 which form opposite ends of each slot. The slots 102 form spaced sections 65 on opposite sides of each slot 102, wherein a first one of the sections comprises one of the projections 64 which extends into the pocket 66 of an opposite second one of the sections 65. In an alternate embodiment a single slot could comprise more than one projection 64 and one pocket 66. Each projection/pocket 64/66 could face forward, or rearward, or multiple projection/pocket 64/66 pairs could face both forward and rearward on the tube. In this example embodiment a pattern of four of the slots 102 is repeated wherein the pattern has each of the four projection/pockets arranged in a general spiral pattern along the length of the tube; about 90 degrees rotated relative to each other.

The portions 67, 69 are aligned generally perpendicular to the center axis 104 of the tube 100, but in an alternate embodiment they could be angled. The pair of projection/pocket 64/66 extend generally parallel to the center axis 104, but in an alternate embodiment they also could be angled relative to the center axis 104. In this example embodiment, as noted above, the pair of projection/pocket 64/66 of one slot is offset by a rotational angle about the axis 104 relative to an adjacent slot 102 by 90 degrees. Thus, a first pair of projection/pocket 64/66 is located at a 0 (zero) degree reference angle, a subsequent second pair of projection/pocket 64/66 is located at a 90 degree reference angle, a subsequent third pair of projection/pocket 64/66 is located at a 180 degree reference angle, a subsequent fourth pair of projection/pocket 64/66 is located at a 270 degree reference angle, and a subsequent fifth pair of projection/pocket 64/66 is located back at a 0 (zero) degree reference angle. Thus, in this embodiment the slots 102 provide a general spiral pattern to the layout of the projection/pocket 64, 66 on the tube 100. In alternate embodiments any suitable amount of rotational angle difference between adjacent projections/pockets 64/66 could be provided, such as 120 degrees or 72 degrees for example. Also, the rotational angle might not be uniform.

Although the embodiment of FIGS. 13-15 have been described with reference to a general rectangular projection 64 and general rectangular pocket 66, the shapes of the projections and pockets could be different. For example, the example shown in FIGS. 13-15 could use shapes such as those shown in FIGS. 8-12. Alternatively, other alternatives shapes could be used for the projections and pockets.

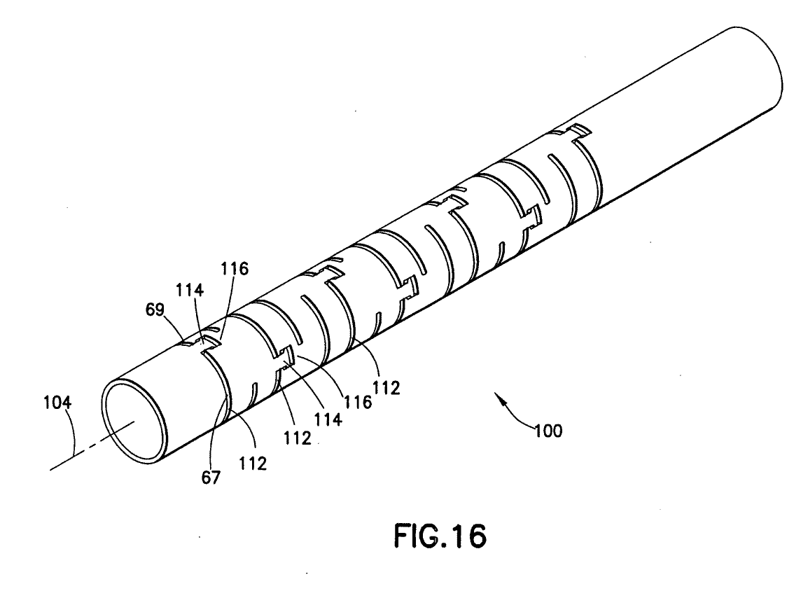

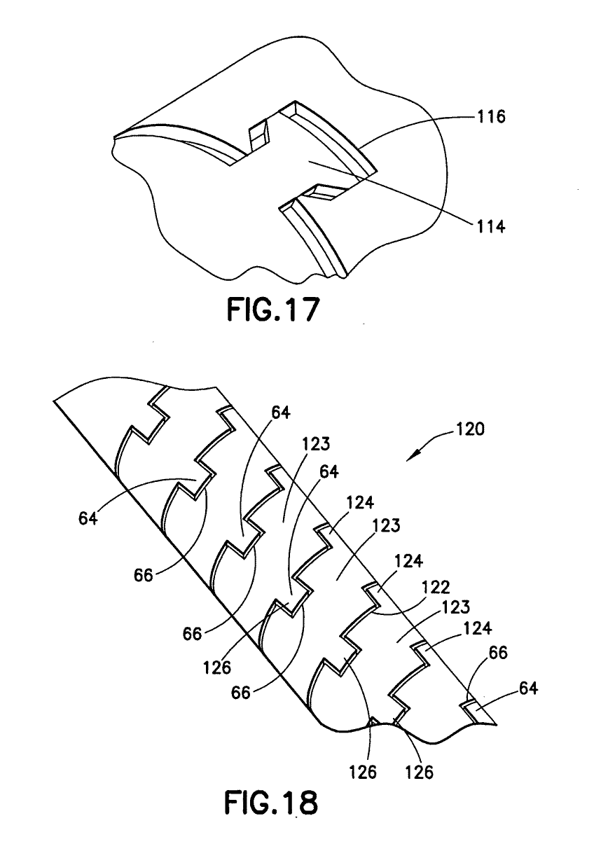

Referring also to FIGS. 16 and 17, another alternate embodiment of the tube used for at least part of the frame of the endoscope 10 is shown. The tube 110 is a one piece member comprised of a suitable material such as plastic, metal or metal alloy for example. The tube 110 has multiple slots 112 therein. Referring also to FIG. 17, each slot 112 forms one projection 114 and one pocket 116 as well as portions 67, 69 on opposite sides of the pair of projection/pocket 114/116. In an alternate embodiment a single slot could comprise more than one projection 114 and one pocket 116. Each projection/pocket 114/116 could face forward, or rearward, or multiple projection/pocket 114/116 could face both forward and rearward on the tube.

The portions 67, 69 are aligned generally perpendicular to the center axis 104 of the tube 100, but in an alternate embodiment they could be angled. The pair of projection/pocket 114/116 extend generally parallel to the center axis 104, but in an alternate embodiment they also could be angled relative to the center axis 104. In this example embodiment the pair of projection/pocket 114/116 of one slot is offset by a rotational angle about the axis 104 relative to an adjacent slot 112 by 120 degrees. Thus, a first pair of projection/pocket 114/116 is located at a 0 (zero) degree reference angle, a subsequent second pair of projection/pocket 114/116 is located at a 120 degree reference angle, a subsequent third pair of projection/pocket 114/116 is located at a 240 degree reference angle, and a subsequent fourth pair of projection/pocket 114/116 is located at a 0 (zero) degree reference angle. Thus, in this embodiment the slots 112 provide a general spiral pattern to the layout of the projection/pocket 114/116 on the tube 100. In alternate embodiments any suitable amount of rotational angle difference between adjacent projections/pockets 114/116 could be provided. Also, the rotational angle might not be uniformly equal; the angles could vary.

The projections 114 in this example embodiment have a general “T” shape. The pockets 116 also have a general “T” shape. In addition to limiting torsional twisting of the tube, the “T” shaped projections/pockets 114/116 can limit axial bending of the tube. The projection is movably located in the pocket to be able to longitudinally move in the pocket, and the projection and the pocket have interlocking shapes to limit longitudinal movement of the projection out from the pocket. In this embodiment, one or more protrusions 114 from one side of a cut are located in cavities 116 on the adjacent side of the cut and act to control the relative motion of the two sides of the cut. They act as an over-travel limiter. This embodiment further improves the reliability of controlling the relative motion of the two sides of the cut by limiting the one or more protrusions from moving outside of their associated cavities when bending the tube. Additionally, this embodiment limits the maximum deflection of the tube in the axial direction since the one or more protrusions can interfere with its associated cavity(ies).

In an alternate embodiment, each of the slots could have a general spiral shape. Each of these slots could have a patterned shape to provide multiple projections 114 and pockets 116 in one or more slots. In one type of alternate embodiment a slot could revolve about the axis 104 more than 360 degrees, such as about two times (720 degrees) for example. In this example embodiment each slot could form two pairs of the projections/pockets 114/116 which are located generally equally spaced about the axis 104, such as about 180 degrees apart. The successive slots could alternate where they start and end such that pairs of the projections/pockets 114/116 are staggered about 90 degrees apart. Portions of adjacent slots could be intermixed or interleaved with one another. The projections/pockets could also have different shapes (they do not need to have the same shape). In one type of alternate embodiment the generally spiral slot might only have one pair of the projections/pockets.

Referring also to FIG. 18, another alternate embodiment of the tube used for at least part of the frame of the endoscope 10 is shown. The tube 120 is a one piece member comprised of a suitable material such as plastic, metal or metal alloy for example. The tube 120 has a section with a single slot 122 therein. The slot 122 has a general spiral pattern revolving about a center longitudinal axis of the tube. The slot 122 is not straight. Instead, the slot has a patterned shape to provide multiple projections 64 and pockets 66. In this example embodiment the slot forms forward projecting pairs 124 of the projections/pockets 64/66 and rearward projecting pairs 126 of the projections/pockets 64/66. The slot 122 provides a general spiral pattern to the layout of the projection/pocket 64/66 on the tube 120.

In this embodiment, one or more protrusions from one side of a single cut are captured in cavities on the adjacent side of the cut and act to control the relative motion of the two sides of the cut. In this embodiment the single cut is represented as a spiral along the length of the tube. In an alternate embodiment the protrusions could be designed to interfere with their cavities similar to that shown in FIG. 17. This over-travel “T” shape limiter can improve the reliability by controlling the relative motion of the two sides of the cut by limiting the one or more protrusions from moving outside of their associated cavities when bending the tube. Additionally, this can limit the maximum deflection of the tube in the axial direction since the one or more protrusions will interfere with their associated cavities. The slot 122 forms spaced sections 123 on opposite sides of the slot 122, wherein a first one of the sections comprises at least one of the projections 64 and/or pockets 66, and at least one opposite second one of the sections 123 comprises at least one respective mating projection 64 and/or pocket 66.

Although the embodiment of FIG. 18 has been described with reference to a general rectangular or square projection 64 and general rectangular or square pocket 66, the shapes of the projections and pockets could be different. For example, the example shown in FIG. 8 could use shapes such as those shown in FIGS. 8-12. Alternatively, other alternatives shapes could be used for the projections and pockets.

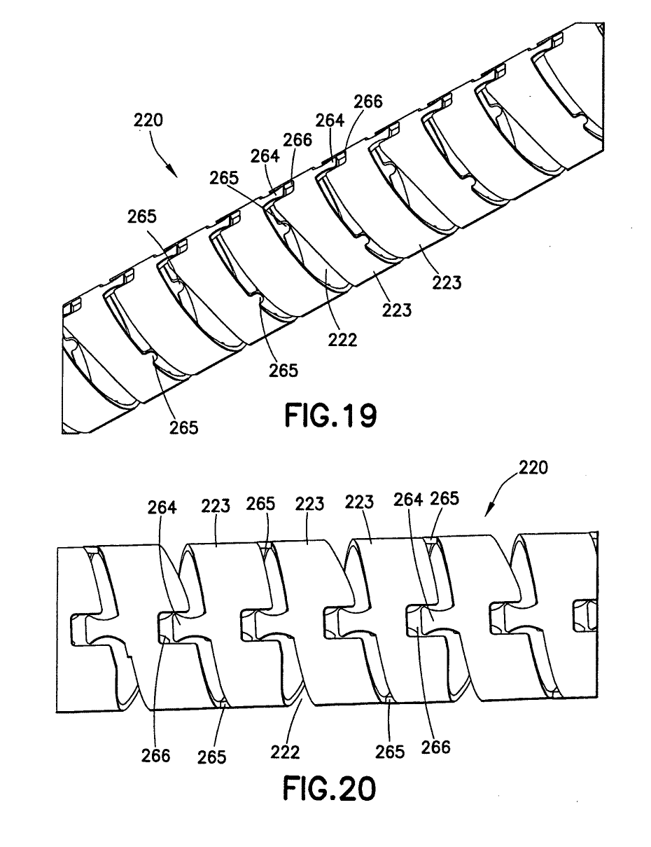

Referring also to FIGS. 19-20 another alternate embodiment of the tube used for at least part of the frame of shat of the endoscope 10 is shown. The tube 220 is a one piece member comprised of a suitable material such as plastic, metal or metal alloy for example. The tube 220 has a section with a single slot 222 therein. The slot 222 has a general spiral pattern revolving about a center longitudinal axis of the tube. The slot 222 is not straight. Instead, the slot has a patterned shape to provide multiple projections 264, 265 and pockets 266. In this example embodiment the pairs of projections/pockets 264/266 function as the torsional over-travel limiter. The projections 265 do not project into any pocket, but instead can contact an opposite side of the slot as a stand-off and bending type of limiter. The slot 222 provides a general spiral pattern to the layout of the projection/pocket 264/266 on the tube 220, and the projections 265 on the tube 220.

In this embodiment, one or more protrusions from one side of a single cut are captured in cavities on the adjacent side of the cut and act to control the relative motion of the two sides of the cut. In this embodiment the single cut is represented as a spiral along the length of the tube. The slot 222 forms spaced sections 223 on opposite sides of the slot 222, wherein a first one of the sections comprises at least one of the projections 264 and/or pockets 266, and at least one opposite second one of the sections 223 comprises at least one respective mating projection 264 and/or pocket 266.

An example embodiment of the invention can provide an endoscope comprising a control section 12; and a shaft 14 extending from the control section 12, wherein the shaft 14 includes a frame comprising a one-piece tube 100, 110, 120, 220 wherein the tube comprises a first slot 102, 112, 122, 222 into the tube along at least one length of the tube, wherein the first slot has a shape which forms multiple projections 64, 114, 246 and respective pockets 66, 116, 266 in the tube with the projections extending into the pockets to form over-travel limiters which are sized and shaped to limit relative motion of sections of the tube relative to each other in at least one direction. The first slot can extend more than 360 degrees about a longitudinal axis 104 of the tube. The first slot can have a general spiral shape about the longitudinal axis of the tube. The pockets and the projections can have general square or rectangular shapes. The slot can form at least three pairs of the projections and pockets. The pairs can be generally equally spaced about the axis of revolution of the first slot. The projections can include a first projection extending in a general forward direction and a second projection extending in a general rearward direction. The projections can each have a general “T” shape. The pockets can each have a general “T” shape. The tube can comprise a plurality of the first slots. The tube can comprises a second slot in the tube which does not have the projections and pockets, wherein the second slot has a revolute path of at least 180 degrees.

An example embodiment of the invention can provide an endoscope shaft frame member comprising a one-piece tube 100, 110, 120, 220 wherein the tube comprises a slot 102, 112, 122, 222 into the tube along one length portion of the tube, wherein the slot has a general spiral shape about a longitudinal axis of the tube along the length, wherein the slot forms a pocket and a projection which is located in the pocket such that the projection and the pocket form an over-travel limiter which is sized and shaped to limit axial twist deformation of the tube. The slot can form multiple pairs of the projection and pocket. The projections can includes a first projection extending in a general forward direction and a second projection extending in a general rearward direction. The slot can extend more than 360 degrees about the longitudinal axis of the tube. The pocket and the projection may have general square or rectangular shapes. The projection can have a general “T” shape. The pocket may have a general “T” shape. The tube may comprise a plurality of the slots.

An example embodiment of the invention can provide a method comprising providing a tube; and making a slot 102, 112, 122 or 222 into the tube to form at least one section of the tube with an increased flexibility, wherein the slot has a shape which forms multiple projections and respective pockets (64/66 or 114/116 or 246/266 for example) in the tube with the projections extending into the pockets to form over-travel limiters which are sized and shaped to limit relative motion of sections of the tube relative to each other in at least one direction.

The example embodiments of the invention do not require a braid to provide the torque stability or column strength. The example embodiments only require a cover to provide a seal between the inside of the shaft and the outside environment. This cover can additionally be significantly thinner than conventional covers since it is not required to provide the function of column strength or torque stability. The overall benefit is a thinner walled shaft with better torque stability and column strength. A proposed embodiment of a shaft frame for use on flexible endoscopes includes a shaft frame made from any resilient material such as plastic or metal. The shaft frame can have a slotted tube with at least one slot on the shaft. The slot or slots may be angled relative to the shaft's longitudinal axis. In the example embodiments, slot(s) are located spirally along an axis of the shaft with (but not limited to) 3 patterns equally circumferentially spaced. Each slot includes at least one protrusion “tab” and at least one mating pocket “notch”. At least one tab interlocks with at least one notch to provide required torque stability. Alternatively, flexibility to bending in different directions may be controlled by changing the axial spacing, depth or shape of particular slots or pattern of cutting a single slot.

A single slot embodiment is not limited to a spiral. The width of each slot, the number of slots and their orientation and axial spacing on the shaft may be designed to provide the minimum required shaft bending radius. In the preferred embodiment, the shaft minimum bend radius decreases with distal distance along the shaft. The bend radius variance may be controlled by both individual slot widths spacing adjustments as well as by spacing patterns of slots along the shaft. The width of each slot and the tab-to-notch axial spacing may be identical and affect the shaft column strength. The slot and tab-to-notch shaft construction has significantly higher axial compression resistance (compared to the spiral shaft construction) with the compression force applied axially to the only one open slot side which is closing.

In the flexible endoscope application, the optimal shaft frame design achieves all of the following objectives:
Improved torque stability with required shaft flexibility
Thinner overall shaft wall
Maximized column strength without compromising shall flexibility

With twisted force applied, the torsion displacement of each slot adjacent section is limited by the side gap between interlocking tab and notch of this slot. With the known gap width (as small as necessary) the shaft total torsion angle is predictable and controlled. For this shaft construction, the wire braid is not required as a structural member, but can be used as a cosmetic element only to cover shaft features (slots and tabs and notches) under the shaft cover tube.

These example embodiments show multiple slots, but similar function can be a achieved with a single cut (not necessarily a spiral) that extends along and around the tube. The tube does not need to have a circular cross section as shown in the figures. The cross section of the tube could be rectangular or oval for example.

In the example embodiment shown in FIG. 18, one end of the slot 122 is located at a different longitudinal length of the tube than the opposite end of that slot. Thus, this example embodiment illustrates that one or more of the slots could be provided which have a first end located at a first longitudinal length of the tube and an opposite second end of that slot at a different longitudinal length. This could be accomplished with a general spiral shape of the slot along a length of the tube. However, in an alternate embodiment the slot might not be a spiral shaped slot; such as a stepped slot or serpentine shaped slot for example. The shape might also be random, so long as the different lengths of the slot (such as its opposite ends for example) are located at different longitudinal locations on the tube.

It should be understood that the foregoing description is only illustrative of the invention. Various alternatives and modifications can be devised by those skilled in the art without departing from the invention. For example, features recited in the various dependent claims could be combined with each other in any suitable combination(s). In addition, features from different embodiments described above could be selectively combined into a new embodiment. Accordingly, the invention is intended to embrace all such alternatives, modifications and variances which fall within the scope of the appended claims.

What is claimed is:

1. An endoscope comprising:
a control section; and
a shaft extending from the control section, wherein the shaft includes a frame comprising a one-piece tube, wherein the tube comprises at least one slot into the tube to form spaced sections on opposite sides of the slot, wherein a first one of the sections comprises at least one projection which extends into at least one pocket of a second one of the sections such that the projection and pocket form an over-travel limiter to limit relative motion of the first and second sections relative to each other in at least one direction.

2. An endoscope as in claim 1 wherein the at least one slot comprises a plurality of slots extending into the tube from different sides of the tube.

3. An endoscope as in claim 1 wherein the at least one direction is an axial twist direction.

4. An endoscope as in claim 1 wherein the projection is configured to slide generally longitudinally forward and backward at least partially in the pocket.

5. An endoscope as in claim 1 wherein lateral sides of the projection are located to contact opposite lateral sides of the pocket when the tube is axially twisted.

6. An endoscope as in claim 1 wherein the at least one slot includes a three dimensional curved general zigzag shape slot.

7. An endoscope as in claim 1 wherein the at least one slot has opposite ends on opposite sides of the tube which are aligned with each other and are arranged generally perpendicular to a center axis of the tube.

8. An endoscope as in claim 1 wherein the at least one slot includes a single slot having a shape which forms multiple ones of the projection and respective pocket in the tube with the projections extending into the pockets.

9. An endoscope as in claim 1 wherein the at least one slot extends more than 360 degrees about a longitudinal axis of the tube.

10. The endoscope as claimed in claim 1 wherein the at least one slot has a general spiral shape about a longitudinal axis of the tube.

11. The endoscope as claimed in claim 1 wherein the slot forms at least one additional projection which extends from the first section and contacts the second section, wherein the at least one additional projection does not extend into a pocket of the second section.

12. The endoscope as claimed in claim 1 wherein the at least one slot has a plurality of the projection and pocket, wherein the plurality of projections are arranged in a general spiral pattern in the tube around a longitudinal axis of the tube.

13. The endoscope as claimed in claim 1 wherein the at least one first slot forms at least two pairs of the projection and pocket.

14. The endoscope as claimed in claim 13 wherein the pairs are generally equally spaced about an axis of revolution.

15. The endoscope as claimed in claim 13 wherein at least two of the pairs are generally non-equally spaced about an axis of revolution.

16. The endoscope as claimed in claim 13 wherein the projections includes a first projection extending in a general forward direction and a second projection extending in a general rearward direction.

17. The endoscope as claimed in claim 1 wherein the projection is movably located in the pocket to be able to longitudinally move in the pocket, and wherein the projection and the pocket have interlocking shapes to limit longitudinal movement of the projection out from the pocket.

18. An endoscope shaft frame member comprising a one-piece tube, wherein the tube comprises at least one slot into the tube, wherein at least one of the slots has a non-straight shape to form at least one projection formed by the slot which extends into at least one pocket formed by the slot such that upon axial twist deformation of the tube the at least one projection is adapted to contact the at least one pocket to form an over-travel limiter to limit the axial twist deformation of the tube.

19. An endoscope shaft frame member as in claim 18 wherein the at least one slot comprises multiple slots extending into the tube from at least two sides of the tube.

20. An endoscope shaft frame member as in claim 18 wherein the at least, one projection is configured to slide generally longitudinally in an arc forward and backward in the at least one pocket.

21. An endoscope shaft frame member as in claim 18 wherein lateral sides, of the at least one projection are spaced from opposite sides of the at least one pocket and are located to contact at least one of the lateral sides of the at least one pocket when the tube is axially twisted.

22. An endoscope shaft frame member as in claim 18 wherein the at least one slot includes a three dimensional curved general zigzag shape slot.

23. An endoscope shaft frame member as in claim 18 wherein the at least one slot forms at least one additional projection which does not extend into a pocket.

24. An endoscope shaft frame member as in claim 18 wherein the at least one slot has opposite ends on opposite sides of the tube which are aligned with each other and are arranged generally perpendicular to a center axis of the tube.

25. An endoscope shaft frame member as in claim 18 wherein the at least one slot has a general spiral shape about a longitudinal axis of the tube.

26. The endoscope shaft frame member as claimed in claim 18 wherein the slot forms multiple pairs of the projection and pocket.

27. The endoscope shaft frame member as claimed in claim 26 wherein the projections includes a first projection extending in a general forward direction and a second projection extending in a general rearward direction.

28. The endoscope shaft frame member as claimed in claim 18 wherein the at least one slot extends more than 360 degrees about a longitudinal axis of the tube.

29. The endoscope shaft frame member as claimed in claim 18 wherein the pocket and the projection have general square or rectangular shapes.

30. The endoscope shaft frame member as claimed in claim 18 wherein the tube comprises a plurality of the slots.

31. The endoscope shaft frame member as claimed in claim 18 wherein the plurality of slots has a plurality of the projections arranged in a general spiral pattern in the tube around a longitudinal axis of the tube.

32. An endoscope comprising:
a control section; and
a shaft extending from the control section, wherein the shaft includes a frame comprising an endoscope shaft frame member as in claim 18.

33. A method comprising:
providing a one-piece tube;
making at least one slot into the tube to form at least one section of the tube with an increased flexibility, wherein the at least one slot comprises a slot having a non-straight shape to form a projection formed by the slot which extends into a pocket formed by the slot such that the projection and pocket form an over-travel limiter to limit axial twist deformation of the tube.

34. A method as in claim 33 wherein the slot is made as a single slot with multiple ones of the projection and pocket provided as pairs.

35. A method as in claim 33 wherein the at least one slot is made as a plurality of the slot having their respective projections and pockets arranged in a general spiral pattern in the tube around a longitudinal axis of the tube.

36. A method as in claim 33 wherein the slot is made with a first end located at a first longitudinal length of the tube and an opposite second end at a different longitudinal length of the tube.
